(19) United States
(12) Patent Application Publication
(10) Pub. No.: US 2009/0253779 A1
(43) Pub. Date: Oct. 8, 2009

(54) Expression of Rbap48 in Memory and Aging and Methods Related Thereto

(76) Inventor: Scott A. Small, New York, NY (US)

Correspondence Address: COOPER & DUNHAM, LLP, 30 Rockefeller Plaza, 20th Floor, NEW YORK, NY, 10112, US

(21) Appl. No.: 12/083,394

(22) PCT Filed: Oct. 19, 2006
(86) PCT No.: PCT/US06/40677

§ 371 Date: May 22, 2009

Related U.S. Application Data
(60) Provisional application No. 60/728,668, filed on Oct. 19, 2005.

(51) Int. Cl. A61K 31/7088; C12P 21/04; C12Q 1/00

(52) U.S. Cl. 514/44 R; 435/71.1; 435/4

(57) ABSTRACT

This invention provides a method for increasing the expression of RbAp48 protein in a eukaryotic cell comprising introducing into the cell an agent which specifically increases the expression of RbAp48 protein in the cell. This invention also provides a method for treating a subject afflicted with age-related memory decline comprising administering to the subject a therapeutically effective amount of an agent which specifically increases the expression of RbAp48 protein in the cells of the subject's brain. This invention further provides a method for determining whether an agent causes an increase in the expression of RbAp48 protein. Finally, this invention provides a method for determining whether an agent causes an increase in the activity of RbAp48 protein.

This invention was made with support under United States Government Grant Nos. AG08702, AG00949, AG009219 from the National Institutes of Health. Accordingly, the United States Government has certain rights in the subject invention.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

Cross-species studies have established that the hippocampal formation is a brain structure targeted by the aging process, contributing to age-related memory decline. The hippocampal formation is a complex circuit made up of molecularly-distinct neuronal populations (Lein et al., 2004), organized into separate but interconnected hippocampal subregions (Amaral and Witter, 1989). Aging causes hippocampal dysfunction by affecting synaptic physiology, with a relative absence of cell death or other histological markers (Gallagher et al., 1996). Because of circuit properties, a defect in a primary subregion will affect physiologic function throughout the hippocampus (Barnes, 1994).

By investigating the hippocampus as a circuit, functional imaging studies in humans, monkeys, and rats have established the spatiotemporal profile of the aging hippocampal formation (Smith et al., 1980; Small et al., 2002; Small et al., 2004). Spatially, functional imaging studies have pinpointed the dentate gyrus as the hippocampal subregion most vulnerable to the effects of advancing age (Small et al., 2004). In contrast, pyramidal-cell subregions such as the entorhinal cortex are relatively spared. Temporally, functional imaging has established that the physiologic integrity of the dentate gyrus declines in a linear fashion across the life-span (Small et al., 2002; Small et al., 2004).

### SUMMARY OF THE INVENTION

This invention provides a method for increasing the expression of RbAp48 protein in a eukaryotic cell comprising introducing into the cell an agent which specifically increases the expression of RbAp48 protein in the cell.

This invention also provides a method for treating a subject afflicted with age-related memory decline comprising administering to the subject a therapeutically effective amount of an agent which specifically increases the expression of RbAp48 protein in the cells of the subject's brain.

This invention further provides a method for determining whether an agent causes an increase in the expression of RbAp48 protein, comprising the steps of (a) contacting the agent with a eukaryotic cell under conditions which, in the absence of the agent, permit expression of RbAp48 protein; (b) determining, after a suitable period of time, the amount of expression of RbAp48 protein in the cell; and (c) comparing the amount of expression determined in step (b) with the amount of expression which occurs in the absence of the agent, whereby an increased amount of expression in the presence of the agent indicates that the agent causes an increase in the expression of RbAp48 protein.

Finally, this invention provides a method for determining whether an agent causes an increase in the activity of RbAp48 protein, comprising the steps of: (a) contacting the agent with a eukaryotic cell under conditions which, in the absence of the agent, permit activity of RbAp48 protein therein; (b) determining, after a suitable period of time, the amount of activity of RbAp48 protein in the cell; and (c) comparing the amount of activity determined in step (b) with the amount of activity of RbAp48 protein which occurs in the absence of the agent, whereby an increased amount of activity in the presence of the agent indicates that the agent causes an increase in the activity of RbAp48 protein.

### BRIEF DESCRIPTION OF THE FIGURES

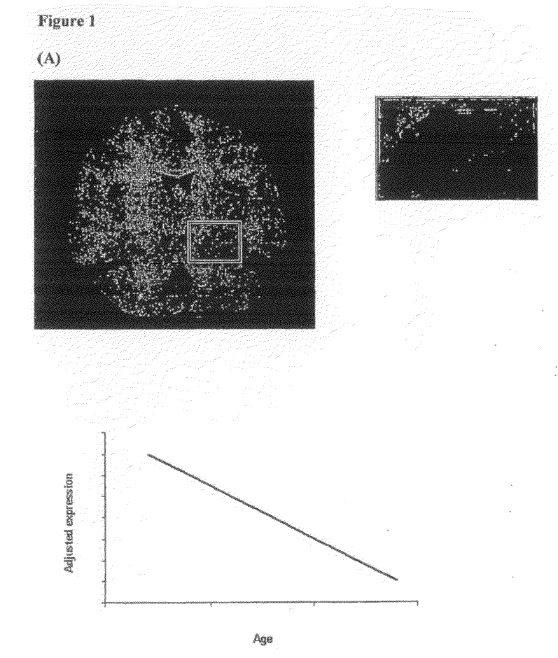

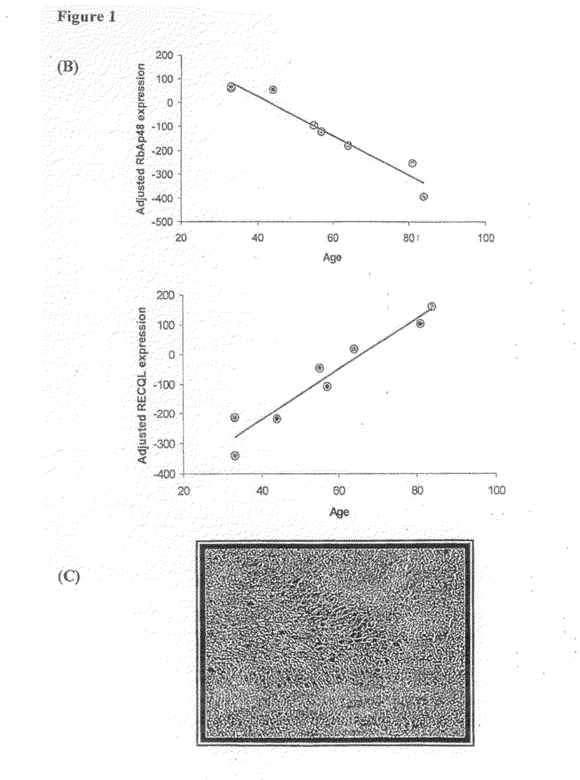

FIG. 1A-C: Expression decline of the transcriptional-regulator RbAp48 best conforms to the spatiotemporal profile of the aging hippocampus.
(A) Functional brain imaging has established the spatiotemporal profile of the aging hippocampal formation. Spatially, the dentate gyrus (color not shown) is differentially vulnerable, while the entorhinal cortex (color not shown) is resistant, to the effects of advancing age. Temporally, the physiologic integrity of the dentate gyrus has been found to decline linearly with age. Accordingly, a molecule that underlies age-related memory decline should be differentially expressed in the dentate gyrus compared to the entorhinal cortex, and adjusted expression levels in the dentate gyrus should either decrease or increase linearly with age (right panel).
(B) Expression decline in RbAp48, a transcriptional-regulator, and expression increase in RECQL, a DNA helicase, conformed to the spatiotemporal profile of the aging hippocampus. Age-related decline in RbAp48 was the primary effect, accounting for the increase in RECQL expression.
(C) Immunocytochemistry demonstrates that RbAp48 is expressed in granule cells of the dentate gyrus.

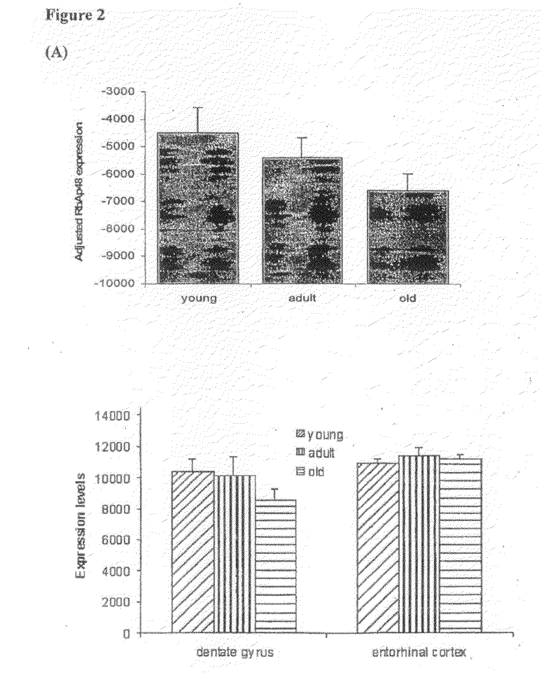

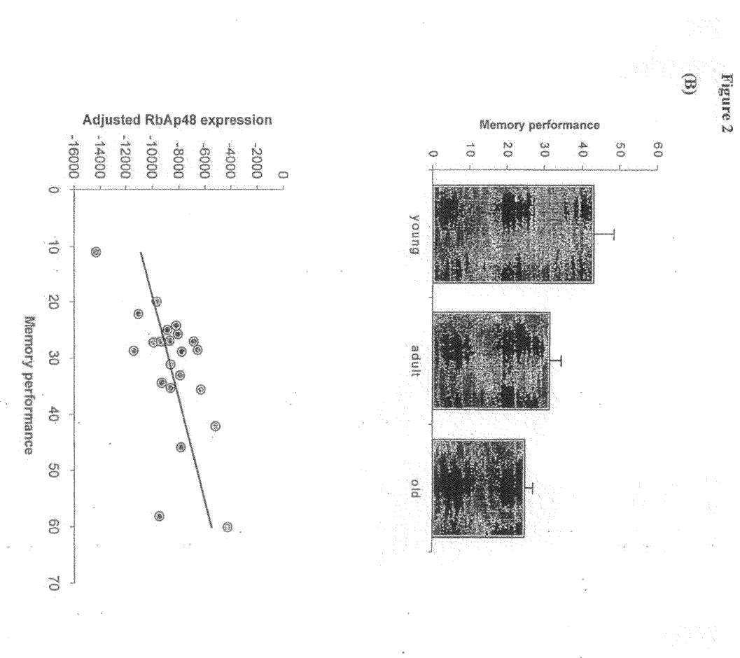

FIG. 2A-B: RbAp48 expression declines in aging rats and correlates with memory function.
(A) RbAp48 expression was measured with Western blots and normalized against actin in both the dentate gyrus and the entorhinal cortex of aging rats. As in humans, changes in dentate gyrus expression, adjusted to each individual's entorhinal cortex, declined linearly with age (left panel). Unadjusted levels are shown from both the dentate gyrus and the entorhinal cortex from all three age-groups (left panel).
B) Memory performance in the Morris water maze, indicated by the time to find an escape platform, declined with age (left panel). Memory performance correlated with dentate gyrus expression, independent of age (right panel).

### DETAILED DESCRIPTION OF THE INVENTION

### Terms

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

As used herein, “agent” shall mean any chemical entity, including, without limitation, a protein, an antibody, a nucleic acid, a small molecule, and any combination thereof. In one embodiment, the agent is known to cross the blood/brain barrier of a mammal (e.g. a human).

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “pharmaceutically acceptable carrier” shall mean any of the various carriers known to those skilled in the art.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

s used herein, “subject” shall mean any animal, such as a primate (e.g. monkey), mouse, rat, guinea pig or rabbit. In the preferred embodiment, the subject is a human.

As used herein, “suitable period of time” shall mean, with respect to the instant assay methods, an amount of time sufficient to permit expression of RbAp48.

As used herein, “therapeutically effective amount” means an amount sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art. In one embodiment, the therapeutically effective amount is from about 1 mg of agent/subject to about 1 g of agent/subject per dosing. In another embodiment, the therapeutically effective amount is from about 10 mg of agent/subject to 500 mg of agent/subject. In a further embodiment, the therapeutically effective amount is from about 50 mg of agent/subject to 200 mg of agent/subject. In a further embodiment, the therapeutically effective amount is about 100 mg of agent/subject. In still a further embodiment, the therapeutically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art.

As used herein, “treating” a disorder shall mean slowing, stopping or reversing the disorder's progression.

### Embodiments of the Invention

This invention provides a method for increasing the expression of RbAp48 protein in a eukaryotic cell comprising introducing into the cell an agent which specifically increases the expression of RbAp48 protein in the cell. The cell may be present, for example, in a cell culture. The cell may be, for example, a brain cell (e.g. a dentate gyrus cell, such as a granule cell or a non-granule cell). The agent may be, for example, a nucleic acid (e.g. an expression vector encoding human RbAp48, and suitable for introduction into human brain tissue as discussed, for example, in U.S. Pat. No. 6,946,126).

This invention also provides a method for treating a subject afflicted with age-related memory decline comprising administering to the subject a therapeutically effective amount of an agent which specifically increases the expression of RbAp48 protein in the cells of the subject's brain. In the preferred embodiment, the subject is human. The agent may be, for example, a nucleic acid (e.g. an expression vector encoding human RbAp48, and suitable for introduction into human brain tissue as discussed, for example, in U.S. Pat. No. 6,946,126).

This invention also provides a method for determining whether an agent causes an increase in the expression of RbAp48 protein, comprising the steps of: (a) contacting the agent with a eukaryotic cell under conditions which, in the absence of the agent, permit expression of RbAp48 protein; (b) determining, after a suitable period of time, the amount of expression of RbAp48 protein in the cell; and (c) comparing the amount of expression determined in step (b) with the amount of expression which occurs in the absence of the agent, whereby an increased amount of expression in the presence of the agent indicates that the agent causes an increase in the expression of RbAp48 protein. In one embodiment, the cell is present in a cell culture. The cell may be, for example, a brain cell (e.g. a dentate gyrus cell, such as a granule cell or a non-granule cell). Determining the amount of expression of RbAp48 protein in the cell can be performed, for example, by determining the amount of RbAp48 protein-encoding mRNA present in the cell, by determining the amount of RbAp48 protein present in the cell, or using an antibody specific for such protein.

This invention further provides a method for determining whether an agent causes an increase in the activity of RbAp48 protein, comprising the steps of: (a) contacting the agent with a eukaryotic cell under conditions which, in the absence of the agent, permit activity of RbAp48 protein therein; (b) determining, after a suitable period of time, the amount of activity of RbAp48 protein in the cell; and (c) comparing the amount of activity determined in step (b) with the amount of activity of RbAp48 protein which occurs in the absence of the agent, whereby an increased amount of activity in the presence of the agent indicates that the agent causes an increase in the activity of RbAp48 protein. In a preferred embodiment, the cell is present in a cell culture. The cell may be, for example, a brain cell (e.g. a dentate gyrus cell, such as a granule cell or a non-granule cell).

This invention is illustrated in the Experimental Details section which follows. This section is set forth to aid in an understanding of the invention but is not intended to, and should not be construed to limit in any way the invention as set forth in the claims which follow thereafter.

### Experimental Details

### Synopsis

Although memory performance declines inexorably with age, the molecular underpinning of this cognitive change remains unknown. This study relies on functional brain imaging to generate a hypothesis-driven model on how molecules associated with memory decline should behave-both spatially and over time. The model was then forward-applied to microarray data generated from human brain tissue ranging the life span. A regionally-selective decline in the expression of RbAp48, a transcriptional-regulator, best conformed to the spatiotemporal model of age-related memory decline. Next, the findings were replicated in a second species, showing a similar pattern of decline in RbAp48 among aging rats. More importantly, RbAp48 expression was found to correlate with memory performance, thus confirming that RbAp48 is associated with memory. Taken together, this study has isolated an unexplored molecular pathway underlying memory and aging.

### Methods

A. Human study

Human Brain Samples: Eight brains ranging from 33-84 years of age, free of histological evidence of brain disease, were obtained at autopsy under a protocol approved by the institution's review board. The dentate gyrus and the entorhinal cortex were identified and sectioned using strict anatomical criteria, following New York Brain Bank procedures. Samples were snap frozen in liquid nitrogen and stored at −80° C.

Gene-expression profiling: For each of the 16 brain samples, total RNA was extracted from entorhinal cortex and dentate gyrus tissue with TRIzol reagent (Invitrogen, Carlsbad, Calif.) and was purified with RNeasy column (Invitrogen). 10 μg total RNA were used to prepare double-stranded cDNA (Superscript, Invitrogen). The T7-(dT)₂₄ primer for cDNA synthesis contained a T7 RNA polymerase promoter site. An in vitro transcription reaction with biotin-labeled ribonucleotides was performed on the cDNA to produce cRNA probes (Bioarray High Yield RNA Transcript Labeling Kit, ENZO Life Sciences, Farmingdale, N.Y.). In the Gene Chip Facility of Columbia University, HG-U133A microarrays (GeneChip, Affymetrix, Santa Clara, Calif.) were hybridized with fragmented cRNA for 16 h in a 45° C. incubator with constant rotation at 60 g. Microarrays were washed and stained on a fluidics station, and scanned using a laser confocal microscope. HG-U133A microarrays were analyzed with Affymetrix Microarray Suite v5.0 and GeneSpring v5.0.3 (Silicon Genetics, Redwood City, Calif.) software, and scaled to a value of 500. Samples which had a 3′/5′ ratio of control genes actin and GAPDH greater than 7, were excluded from analysis. Transcripts whose detection levels had a p-value greater than 0.05 were excluded.

Immunocytochemistry: Coronal blocks of human hippocampal formation were frozen-sectioned using a Microm cryostat at 8 μm thickness. Tissue was either directly quick-frozen or, in some cases, fixed 4% paraformaldehyde in PBS for 18 hr, cryoprotected in 25% sucrose in PBS, and then quick-frozen. Sections on slides were postfixed with 4% paraformaldehyde in PBS, washed with PBS, then treated with 3% H₂O₂, washed, and preincubated for 1 hr in Block solution consisting of 2% horse serum (Vector, Burlingame, Calif.), 1% bovine serum albumin (Sigma-Aldrich Chemical Co., St Louis, Mo.) and 0.1% Triton X-100 (Sigma) in PBS. Slides were then incubated 18 hr at 4° C. in diluted (1:200 in Block solution) polyclonal antiserum to RbAp48. After washing with PBS, immunoreactivity was detected by an avidin-biotin linked peroxidase method, using successive incubations and washes with goat anti-rabbit biotinylated IgG, Vectastain ABC-Elite reagent (Vector), and diaminobenzidine (Sigma) chromogen reagent. Sections were dehydrated and mounted using Permount (Fisher Scientific, Pittsburgh, Pa.).

Data analysis: Microarray data were analyzed based on brain imaging findings in a hypothesis-driven manner: Spatially, ‘aging’ molecules are predicted to be differentially expressed in the dentate gyrus compared to the entorhinal cortex. Temporally, the expression levels of these molecules are predicted to change linearly across the life-span (FIG. 1). In accordance with this hypothesis, a linear regression model was constructed where age and dentate gyrus expression levels were included as primary variables, and entorhinal expression was included as a covariate. Adjusting for entorhinal expression controls inter-individual sources of noise, such as global differences in genetic heritage and environment (Pierce and Small, 2004).

B. Rat study

Subjects: Three groups of rats were investigated: Six young rats 5-months of age, 6 adult rats 12-months of age, and 10 old rats ranging from 23-27 months of age. The Fisher (F-344) rats were obtained from the National Institutes on Aging rodent colony at Harlan (St. Louis, Mo.).

Cognitive testing: All rats were tested with a version of the spatial and cued version of the Morris water maze. A circular tank (183 cm diameter, 43 cm high) was placed in a 2.3×2.7×2.5 m room containing proximal and distal cues. The tank was filled to a depth of 36 cm with water made opaque with the addition of non-toxic white paint (Crayola, Easton, Pa.). For the spatial version of the task, an escape platform was (11.5 cm diameter) was submerged 2.5 cm under the water surface in the northwest quadrant of the tank.

Rats received 6 training trials per day, in blocks of 2 trials, for 4 days. Before the first trial, each rat was positioned onto the escape platform for 30 seconds. Trials were begun by placing each rat into the water, facing the tank wall, from one of 7 pseudo-randomized positions. Following this release, the animal was then given 60 seconds to find the submerged platform, followed by a 30 second rest on the platform. On the 4^{th} testing day following the last spatial trial, a single probe trial was given. During this trial, the escape platform was removed and all rats swam for 60 seconds. The rats were then allowed to rest for a 2-hour period in the incubator. At the completion of this break, each rat was then given 6 trials on the visual discrimination version of the task. During these trials, the water level was lowered so that the escape platform was 2 cm above the surface. An additional Styrofoam cue was hung 30 cm above the platform. On the 5^{th} and final testing day, all animals received an additional 6 visible-cued trials. Both the platform location and rat release location were pseudo-randomized from trial to trial. An overhead video camera connected to a VP114 tracking unit (HVS Image, England) was used to track path-lengths.

Post-mortem dissection: Four days after cognitive testing, rats were anesthetized with isoflurane, were guillotined, and the removed brains were immediately placed in ice-cold artificial cerebro-spinal fluid (ACSF). The dentate gyrus and the entorhinal cortex were resected, transferred to individual micro-centrifuge vials and immediately frozen in liquid nitrogen. The tissue was then held for 4 days at −70 degrees Celsius and was shipped to Columbia University on dry ice for Western blot processing.

Subregional Fractionation and Western Blotting: Frozen rat hippocampal sections of dentate gyrus and entorhinal cortex were soaked in 5 volumes of solution (0.32M Sucrose, 0.5 mM CaCl₂, 1 mM MgCl₂, 1 mM NaHCO₃) supplemented with protease Inhibitor cocktail (Roche) for 15-30 minutes. Samples were homogenized on ice with 12 strokes at 900 rpm using a motor-operated Tephlon-pestle homogenizer. Homogenate was centrifuged at 240× g for 10 min at 4° C., and the supernatant was saved (S1). Western blotting was performed on 3 μg of protein sample (S1) using a 1:2000 dilution of rabbit anti-RbAp48 antibody (Affinity BioReagents).

Data analysis: First, to confirm human findings, a linear regression was performed, where age and dentate gyrus expression levels of RbAp48 were included as primary variables, and entorhinal expression of RbAp48 was included as a covariate. More importantly, to test for an association between RbAp48 expression and memory, a second regression analysis was performed, which included dentate gyrus expression and memory performance as the primary variables, and entorhinal cortex and age as the covariates.

### Results

### A. Human Study

Two molecules from the microarray dataset best conformed to the spatiotemporal model of the aging hippocampus, both involved in DNA metabolism. Expression of RbAp48 a molecule involved in transcriptional-regulation (Henikoff, 2003) declined with age (beta=−0.76, p=0.0004); while expression of RECQL a member of the DNA helicase family (Nakayama, 2002) increased with age (beta=1.35, p=0.014) (FIG. 1). A secondary step-wise regression analysis, where both molecules were assessed simultaneously, revealed that RbAp48 decline was the primary effect, accounting for the increase in RECQL expression. Immunocytochemistry using anti-RbAP48 antibodies confirmed that that RbAp48 is expressed in granule cells of the dentate gyrus (FIG. 1).

### B. Rat Study

To replicate the RbAp48 findings in a second species, and more importantly, to test whether RbAp48 is relevant to memory function, rats who were behaviorally-characterized with the Morris water maze (Gallagher et al., 1993) were investigated. In agreement with prior studies, memory performance declined with age (beta=−0.67, p=0.001) (FIG. 2). As in humans, RbAp48 expression in the dentate gyrus declined in a linear fashion across the life-span (beta=−0.42, p=0.029) (FIG. 2). Remarkably, RbAp48 expression was found to correlate with memory performance (beta=5.2, p=0.006), and notably this correlation is independent of age (FIG. 2). Thus, expression of the transcriptional-regulator RbAp48 is associated with memory performance and undergoes age-related decline in both humans and mice.

### Discussion

Microarray is a technique that, in principle, is well suited to identify molecules underlying changes in brain function (Mirnics et al., 2001; Pongrac et al., 2002; Dobrin and Stephan, 2003). The exploratory power of microarray—the ability to simultaneously compare the expression of thousands of genes—is also its main analytic liability (Mirnics and Pevsner, 2004). Distinguishing meaningful from false-positive expression differences, which naturally occur with multiple comparisons, is one of the main analytic challenges of microarray (Slonim, 2002). This challenge is heightened when investigating physiological changes of the brain (Pongrac et al., 2002), a key characteristic of age-related cognitive decline. Typically, only subtle changes in gene expression are required to affect neuronal function, and so large expression differences, found for example in cancer cells, cannot be relied on to isolate meaningful gene products (Wurmbach et al., 2002). Furthermore, because tissue is typically harvested from post-mortem samples, sources of noise are likewise more extensive. These factors extend beyond the individual genetic and environmental differences that affect any comparison, but also include individual differences in the dying process-a particularly strong influence of expression patterns (Harrison et al., 1995). Thus, low sources of signal and large sources of noise account for the difficulty in pinpointing which among a list of possible molecules directly underlies brain dysfunction.

Combining functional brain imaging with microarray is an approach that can partly address the analytic challenges inherent to microarray (Pierce and Small, 2004). By generating an a priori hypothesis that predicts the manner in which a candidate molecule should behave, imaging findings can be harnessed to enhance the analytic power of microarray (Pierce and Small, 2004). So, as illustrated in this study of cognitive aging, instead of performing microarray on the whole hippocampus formation-whose multiple neuronal populations will contribute to inter-regional sources of variance (Zhao et al., 2001)—imaging findings directed the focus on the dentate gyrus as the primary site of dysfunction. Furthermore, brain imaging has identified the entorhinal cortex as the neighboring subregion most resistant to aging, and using microarray data from this control subregion constrains inter-individual sources of variance. Finally, the imaging-derived temporal profile of dentate gyrus dysfunction was used to further filter microarray data against potentially false-positive findings (Pierce and Small, 2004).

The results of this study have isolated a transcriptional-regulator whose expression is associated with memory performance, and whose age-related decline best correlates with hippocampal dysfunction in both humans and rats. All organ systems contain cells particularly sensitive to the effects of advancing age, and as a general molecular mechanism transcription-regulation has been implicated in cellular aging (Guarente and Kenyon, 2000). Nevertheless, most aging studies have focused primarily on SIR2, which governs transcriptional-regulation through a completely separate molecular pathway (Guarente, 2001). Moreover, to date, transcriptional-regulation has been implicated in age-related dysfunction only in non-neuronal cell lines.

RbAp48 is established as the first transcriptional-regulator implicated in the aging brain. The decline in RbAp48 is not diffuse; among the different neuronal populations that make up the hippocampal circuit, RbAp48 expression declines in hippocampal neurons most sensitive to the effects of advancing age (Smith et al., 1980; Small et al., 2004). A growing number of studies have focused on RbAp48 and its cross-species orthologues (Verreault et al., 1996; Kadonaga, 1998; Hennig et al., 2003), which regulates transcription through various mechanisms (Zhang et al., 2000; Henikoff, 2003).

By combining imaging with microarray, a molecular pathway relevant to memory and aging in humans and rats was isolated, thereby enhancing the understanding of the insidious age-related slide into forgetfulness.

### REFERENCES

1. Amaral D G, Witter M P (1989) The three-dimensional organization of the hippocampal formation: a review of anatomical data. Neuroscience 31:571-591.
2. Barnes C A (1994) Normal aging: regionally specific changes in hippocampal synaptic transmission. Trends Neurosci 17:13-18.
3. Dobrin S E, Stephan D A (2003) Integrating microarrays into disease-gene identification strategies. Expert Rev Mol Diagn 3:375-385.
4. Gallagher M, Burwell R, Burchinal M (1993) Severity of spatial learning impairment in aging: development of a learning index for performance in the Morris water maze. Behav Neurosci 107:618-626.
5. Gallagher M, Landfield P W, McEwen B, Meaney M J, Rapp P R, Sapolsky R, West M J (1996) Hippocampal neurodegeneration in aging. Science 274:484-485.
6. Guarente L (2001) SIR2 and aging—the exception that proves the rule. Trends Genet 17:391-392.
7. Guarente L, Kenyon C (2000) Genetic pathways that regulate ageing in model organisms. Nature 408:255-262.
8. Harrison P J, Heath P R, Eastwood S L, Burnet P W, McDonald B, Pearson R C (1995) The relative importance of premortem acidosis and postmortem interval for human brain gene expression studies: selective mRNA vulnerability and comparison with their encoded proteins. Neurosci Lett 200:151-154.
9. Henikoff S (2003) Versatile assembler. Nature 423:814-815, 817.
10. Hennig L, Menges M, Murray J A, Gruissem W (2003) Arabidopsis transcript profiling on Affymetrix GeneChip arrays. Plant Mol Biol 53:457-465.
11. Kadonaga J T (1998) Eukaryotic transcription: an interlaced network of transcription factors and chromatin-modifying machines. Cell 92:307-313.
12. Lein E S, Zhao X, Gage F H (2004) Defining a molecular atlas of the hippocampus using DNA microarrays and high-throughput in situ hybridization. J Neurosci 24:3879-3889.
13. Mirnics K, Pevsner J (2004) Progress in the use of microarray technology to study the neurobiology of disease. Nat Neurosci 7:434-439.
14. Mirnics K, Middleton F A, Lewis D A, Levitt P (2001) Analysis of complex brain disorders with gene expression microarrays: schizophrenia as a disease of the synapse. Trends Neurosci 24:479-486.
15. Nakayama H (2002) RecQ family helicases: roles as tumor suppressor proteins. Oncogene 21:9008-9021.
16. Pierce A, Small S (2004) Combining brain imaging with microarray: Isolating molecules underlying physiologic disorders of the brain. Neurochem Res 29:1145-1152.
17. Pongrac J, Middleton F A, Lewis D A, Levitt P, Mirnics K (2002) Gene expression profiling with DNA microarrays: advancing our understanding of psychiatric disorders. Neurochem Res 27:1049-1063.
18. Slonim D K (2002) From patterns to pathways: gene expression data analysis comes of age. Nat Genet 32 Suppl: 502-508.
19. Small S A, Tsai W Y, DeLaPaz R, Mayeux R, Stern Y (2002) Imaging hippocampal function across the human life span: is memory decline normal or not? Ann Neurol 51:290-295.
20. Small S A, Chawla M K, Buonocore M, Rapp P R, Barnes C A (2004) From The Cover: Imaging correlates of brain function in monkeys and rats isolates a hippocampal subregion differentially vulnerable to aging. Proc Natl Acad Sci U S A 101:7181-7186.
21. Smith C B, Goochee C, Rapoport S I, Sokoloff L (1980) Effects of ageing on local rates of cerebral glucose utilization in the rat. Brain 103:351-365.
22. Verreault A, Kaufman P D, Kobayashi R, Stillman B (1996) Nucleosome assembly by a complex of CAF-1 and acetylated histones H3/H4. Cell 87:95-104.
23. Wurmbach E, Gonzalez-Maeso J, Yuen T, Ebersole B J, Mastaitis J W, Mobbs C V, Sealfon S C (2002) Validated genomic approach to study differentially expressed genes in complex tissues. Neurochem Res 27:1027-1033.
24. Zhang Q, Vo N, Goodman R H (2000) Histone binding protein RbAp48 interacts with a complex of CREB binding protein and phosphorylated CREB. Mol Cell Biol 20:4970-4978.
25. Zhao X, Lein E S, He A, Smith S C, Aston C, Gage F H (2001) Transcriptional profiling reveals strict boundaries between hippocampal subregions. J Comp Neurol 441:187-196.

What is claimed is:

1. A method for increasing the expression of RbAp48 protein in a eukaryotic cell comprising introducing into the cell an agent which specifically increases the expression of RbAp48 protein in the cell.

2. The method of claim 1, wherein the cell is present in a cell culture.

3. The method of claim 1, wherein the cell is a brain cell.

4. The method of claim 3, wherein the brain cell is a dentate gyrus cell.

5. The method of claim 4, wherein the dentate gyrus cell is a granule cell.

6. The method of claim 4, wherein the dentate gyrus cell is a non-granule cell.

7. The method of claim 1, wherein the agent is a nucleic acid.

8. The method of claim 7, wherein the nucleic acid is an expression vector encoding RbAp48.

9. A method for treating a subject afflicted with age-related memory decline comprising administering to the subject a therapeutically effective amount of an agent which specifically increases the expression of RbAp48 protein in the cells of the subject's brain.

10. The method of claim 9, wherein the subject is human.

11. The method of claim 9, wherein the agent is a nucleic acid.

12. The method of claim 11, wherein the nucleic acid is an expression vector encoding RbAp48.

13. A method for determining whether an agent causes an increase in the expression of RbAp48 protein, comprising the steps of:
(a) contacting the agent with a eukaryotic cell under conditions which, in the absence of the agent, permit expression of RbAp48 protein;
(b) determining, after a suitable period of time, the amount of expression of RbAp48 protein in the cell; and
(c) comparing the amount of expression determined in step (b) with the amount of expression which occurs in the absence of the agent, whereby an increased amount of expression in the presence of the agent indicates that the agent causes an increase in the expression of RbAp48 protein.

14. The method of claim 13, wherein the cell is present in a cell culture.

15. The method of claim 13, wherein the cell is a brain cell.

16. The method of claim 15, wherein the brain cell is a dentate gyrus cell.

17. The method of claim 16, wherein the dentate gyrus cell is a granule cell.

18. The method of claim 16, wherein the dentate gyrus cell is a non-granule cell.

19. The method of claim 13, wherein determining the amount of expression of RbAp48 protein in the cell is performed by determining the amount of RbAp48 protein-encoding mRNA present in the cell.

20. The method of claim 13, wherein determining the amount of expression of RbAp48 protein in the cell is performed by determining the amount of RbAp48 protein present in the cell.

21. The method of claim 13, wherein determining the amount of RbAp48 protein in the cell is performed using an antibody specific for such protein.

22. A method for determining whether an agent causes an increase in the activity of RbAp48 protein, comprising the steps of:
(a) contacting the agent with a eukaryotic cell under conditions which, in the absence of the agent, permit activity of RbAp48 protein therein;
(b) determining, after a suitable period of time, the amount of activity of RbAp48 protein in the cell; and
(c) comparing the amount of activity determined in step (b) with the amount of activity of RbAp48 protein which occurs in the absence of the agent, whereby an increased amount of activity in the presence of the agent indicates that the agent causes an increase in the activity of RbAp48 protein.

23. The method of claim 22, wherein the cell is present in a cell culture.

24. The method of claim 22, wherein the cell is a brain cell.

25. The method of claim 24, wherein the brain cell is a dentate gyrus cell.

26. The method of claim 25, wherein the dentate gyrus cell is a granule cell.

27. The method of claim 25, wherein the dentate gyrus cell is a non-granule cell.
